(19) United States
(12) Patent Application Publication
(10) Pub. No.: US 2005/0203329 A1
(43) Pub. Date: Sep. 15, 2005

(54) Dispensing and injection system for radiopharmaceuticals

(76) Inventors: Akio Muto, Shonandai Fujisawa-City (JP); Chi-Min Hu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/044,176

(22) Filed: Jan. 28, 2005

(30) Foreign Application Priority Data
Feb. 6, 2004 (TW) 93102751

(51) Int. Cl. A61M 36/14; A61K 51/00

(52) U.S. Cl. 600/1

(57) ABSTRACT

Disclosed is a dispensing and injection system for radiopharmaceuticals, wherein a dosage calibrator is arranged inside a radiation-shielded hermetic chamber for detecting the radioactivity of radiopharmaceuticals contained in a vial. At least one saline water cartridge is arranged inside the casing and includes an internal passage, radiopharmaceuticals discharge end, a radiopharmaceuticals injection end, a and saline water reservoir outlet end. The saline water cartridge forms a saline water reservoir. A movable dispensing and injection mechanism controls radiopharmaceuticals dispensing operation of a syringe and controls movement of the syringe between a radiopharmaceuticals dispensing position and an injection position. When the movable dispensing and injection mechanism moves the syringe to the radiopharmaceuticals dispensing position, the syringe withdraws a predetermined amount of radiopharmaceuticals from the vial. When moved to the injection position, the syringe injects the withdrawn radiopharmaceuticals into the radiopharmaceuticals injection end of the saline water cartridge. After the radiopharmaceuticals is injected into a patient, the saline water stored in the saline water reservoir of the saline water cartridge is withdrawn for effecting flushing process.

### FIELD OF THE INVENTION

The present invention relates generally to a medicine dispensing and injection system, and in particular to radiation-shielded dispensing and injection system for radiopharmaceuticals.

### BACKGROUND OF THE INVENTION

Due to the unique invivo imaging capability, positron emission tomography (PET) has been recently used in early detection and treatment of cancers that could not be detected previously. This makes PET one of most important measures for diagnosis of a variety of tumors, as well as the main stream of future nuclear medicine

Positron radio-nuclides (radiopharmaceuticals) for PET are generated in a cyclotron, which are then composed with other elements to form compound/molecules, such as glucose, amino acid, and water by radio-chemist and conveyed to injection room for injection into human body by medical employees in order to carry out PET diagnosis by doctors. The positrons are annihilated with electrons inside the human body, which emits gamma rays running in opposite directions, which can be detected by PET and, after been processed by a computer system for imaging, provides functional images and parameters for diagnosis.

The PET facility is of great help for medical diagnosis, but the positron radionuclides of the PET give off strong radiation. Thus, it is a major challenge to protect the radio-chemists and medical employees from over-exposure to radiation.

During the PET process, the radio-chemists, doctors, nurses, and medical assistants must do quantity measurement, quality control, dispensing, conveyance, and injection of the radiopharmaceuticals. If they are not properly protected from radiation, then their health is subject to serious hazard.

In the hospitals that employ PET for diagnosis, in order to effect radiation protection, a conventional way is taken, which, after the radiopharmaceuticals is filled into a vial, disposes the vial into a lead canister closed by a lid for conveyance. However, even the radiopharmaceuticals is accommodated in a lead canister, medical employees are still subject to radiation during the process of retrieving, disposing, quantity measurement, quality control and injection of the radiopharmaceuticals. Further, since the radiation energy of PET radiopharmaceuticals is very high, a very heavy lead canister must be used, which causes a serious burden to the medical employees.

Although a variety of conventional devices are designed to control radiation contamination of the radiopharmaceuticals, these devices are only of limited effectiveness. For example, the conventional devices are of designs focusing on the structure of the canister for eliminating leakage of the radiopharmaceuticals. However, in practical applications, an operator for retrieval, disposition, quantity measurement, quality control, and injection of the radiopharmaceuticals is still subject to great risk of radiation contamination by the radiopharmaceuticals.

Due to the fact that the conventional designs for the canister are insufficient to protect the medical employees, radiopharmaceuticals dispensing equipments and injection equipments of different functions are available. Among the currently commercial injection equipments, stand-alone automatic injection equipment and dispensing equipment are available. These equipments, however, still cause risk of exposure to the radiation energy of the radiopharmaceuticals when the medical employees retrieve the radiopharmaceuticals, dispose the radiopharmaceuticals into the canister, convey the radiopharmaceuticals to the injection equipment, and position the radiopharmaceuticals into the injection equipment.

To solve the problem, a system comprising a tube connecting between a dispensing equipment and an injection equipment is available. However, such a system cannot prevent back flow of a patient's blood through the tube into the injection system.

Thus, it is desired to provide a radiopharmaceuticals dispensing and injection system that is practical, safe, and easy to operate in order to overcome the above-discussed problems.

### SUMMARY OF THE INVENTION

A primary objective of the present invention is to provide a radiation-shielded dispensing and injection system for radiopharmaceuticals, which overcomes over-exposure of radiation during the process of radiopharmaceuticals dispensing and injection by performing measurement, dispensing, and injection of the radiopharmaceuticals without the intervention of operators so as to reduce the risk of radiation exposure of the operators and effecting excellent radiation protection.

Another objective of the present invention is to provide a dispensing and injection system for radiopharmaceuticals that prevents contamination of the system by back flow of patent's blood by disposing of used syringes after the system completes the injection operation thereby completely eliminating the problem of back flow of patient's blood to the vial and the system.

The solution of the present invention to overcome the problems of the prior art is that a dosage calibrator is arranged inside a radiation-shielded hermetic chamber for detecting the radioactivity of radiopharmaceuticals contained in a vial. At least one saline water cartridge is arranged inside the casing and includes an internal passage, radiopharmaceuticals discharge end, a radiopharmaceuticals injection end, a and saline water reservoir outlet end. The saline water cartridge forms a saline water reservoir. A movable dispensing and injection mechanism controls radiopharmaceuticals dispensing operation of a syringe and controls movement of the syringe between a radiopharmaceuticals dispensing position and an injection position. When the movable dispensing and injection mechanism moves the syringe to the radiopharmaceuticals dispensing position, the syringe withdraws a predetermined amount of radiopharmaceuticals from the vial. When moved to the injection position, the syringe injects the withdrawn radiopharmaceuticals into the radiopharmaceuticals injection end of the saline water cartridge.

Preferably, after injection of the radiopharmaceuticals into a patient, the saline water stored in the saline water reservoir of the saline water cartridge is withdrawn for effecting flushing process.

Thus, compared to the prior art, the present invention effectively overcomes the problems of inconvenience and poor safety occurring in handling radiopharmaceuticals whereby the radiopharmaceuticals dispensing and injection system in accordance with the present invention ensures excellent radiation protection in moving, measuring, dispensing, handling, injecting the radiopharmaceuticals and is easy to effect automatic control.

### BRIEF DESCRIPTION OF THE DRAWINGS

The present invention will be apparent to those skilled in the art by reading the following description of a preferred embodiment thereof, with reference to the attached drawings, in which:

FIG. 1 is a front-side perspective view of a radiopharmaceuticals dispensing and injection system constructed in accordance with the present invention;

FIG. 2 is an enlarged partial perspective view, illustrating the condition when a vial of the present invention is handled by a robotic manipulating clamp to move into a dosage calibration container and the dosage calibration container, together with the vial, is moved into a dosage calibrator;

FIG. 3 is an enlarged partial perspective view, illustrating the condition when the vial is moved into a vial container after the dosage calibration performed in FIG. 2;

FIG. 4 is an enlarged partial perspective view, illustrating the arrangement among a disposable syringe module, a movable dispensing and injection mechanism, and the vial container;

FIG. 5 is an enlarged partial perspective view, illustrating the condition when a disposable syringe is held by a pick-up arm but a needle atop the syringe does not penetrate into a bottom of the vial yet;

FIG. 6 is an enlarged partial perspective view, illustrating the condition when the disposable syringe is held by the pick-up arm and the needle atop the syringe penetrates into the bottom of the vial, but a plunger of the disposable syringe is not pulled downward yet;

FIG. 7 is an enlarged partial perspective view, illustrating the condition when the disposable syringe is held by the pick-up arm, the needle atop the syringe penetrates into the bottom of the vial, and the plunger of the disposable syringe is pulled downward;

FIG. 8 is a cross-sectional view of a two-open-end structure of the vial employed in the present invention;

FIG. 9 is an enlarged partial perspective view, illustrating the condition when the disposable syringe is moved to an injection position but the syringe needle does not penetrate into a radiopharmaceuticals injection end of a saline water cartridge yet;

FIG. 10 is an enlarged partial perspective view, illustrating the condition when the disposable syringe is moved to an injection position and the syringe needle penetrates into the radiopharmaceuticals injection end of the saline water cartridge;

FIG. 11 is an enlarged partial perspective view, illustrating the condition when the disposable syringe is moved to an injection position, the syringe needle penetrates into the radiopharmaceuticals injection end of the saline water cartridge, and the syringe plunger is pushed upward;

FIG. 12 is a cross-sectional view, illustrating the condition when the needle of the disposable syringe penetrates into the radiopharmaceuticals injection end of the saline water cartridge and the plunger is pushed upward;

FIG. 13 is a cross-sectional view, illustrating the condition when the needle of the disposable syringe penetrates into the radiopharmaceuticals injection end of the saline water cartridge and the plunger is pulled downward;

FIG. 14 is a rear-side perspective view of the radiation-shielded dispensing and injection system in accordance with the present invention; and

FIG. 15 is a perspective view, illustrating the radiation-shielded radiopharmaceuticals dispensing and injection system in accordance with the present invention comprising a withdrawable carrier, a vial access opening zone, and a door.

### DETAILED DESCRIPTION OF PREFERRED EMBODIMENTS

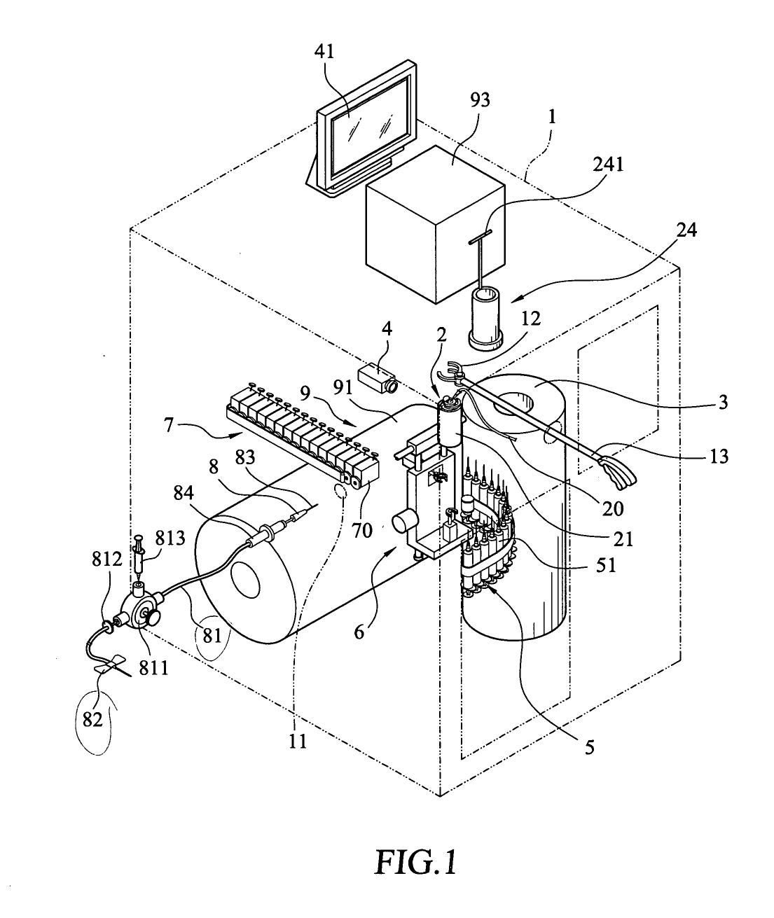

With reference to FIG. 1, which shows a front-side perspective view of a radiation-safe radiopharmaceuticals dispensing and injection system constructed in accordance with the present invention, as shown in the drawing, the present invention comprises a radiation-shielded hermetic chamber 1, which is made of materials having radiation-shielding function, such as lead and tungsten. The radiation-shielded hermetic chamber 1 provides a closed, radiation-shielded interior space, and the hermetic chamber is provided with at least one injection needle insertion hole 11.

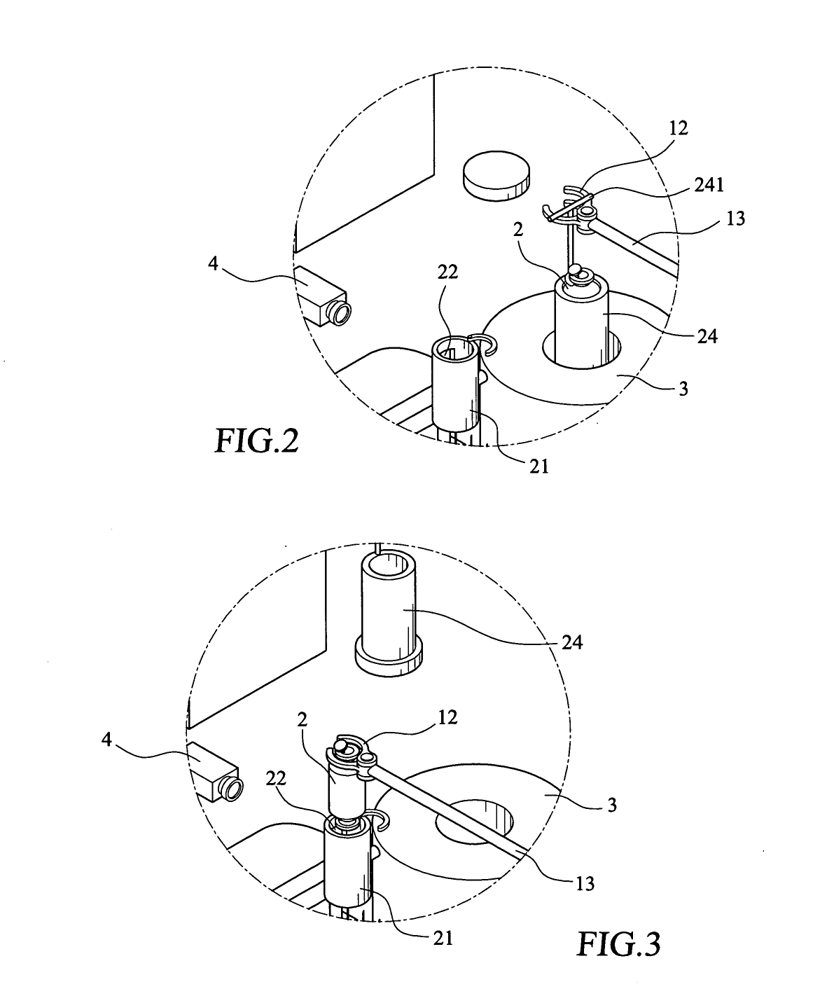

The radiation-shielded hermetic chamber 1 receives a vial 2 (also referring to FIGS. 2 and 3), which contains radiopharmaceuticals. The vial 2 can be handled by a robotic manipulating clamp 12 to move into a vial container 21. The manipulating clamp 12 comprises an extension bar 13, which allows the manipulating clamp 12 to extend into the hermetic chamber 1 and serve as a tool for manual operation. Of course, the manipulating clamp can be completely replaced by an automatic robotic arm.

In a side wall of the vial container 21, a radiopharmaceuticals level monitoring window 22 (see FIG. 2) is formed. A radiopharmaceuticals level monitoring device 4 (such as a charge-coupled device (CCD) based monitor) is arranged at a position adjacent to a position facing the radiopharmaceuticals level monitoring window 22 and the radiopharmaceuticals level monitoring device 4 is connected to a monitor display 41 or a computer device. When a radiopharmaceuticals supply tube 20 supplies radiopharmaceuticals into the vial 2, the monitor display 41 monitors the level of the radiopharmaceuticals. When the radiopharmaceuticals inside the vial 2 reaches a predetermined level, the radiopharmaceuticals supply tube 20 is removed.

After the vial 2 is filled with radiopharmaceuticals, a dosage calibration process is carried out, as illustrated in FIG. 2. During this process, the robotic manipulating clamp 12 moves the vial 2 from the vial container 21 to a dosage calibration container 24. After the vial 2 is positioned in the dosage calibration container 24, the robotic manipulating clamp 12 holds and lifts a T-shaped handle bar 241 of the dosage calibration container 24 to move the dosage calibration container 24, together with the vial 2, into a dosage calibrator 3. The dosage calibrator 3 functions to measure the radioactivity of the radiopharmaceuticals contained in the vial 2.

Referring to FIG. 3, after the dosage calibration process is completed, the robotic manipulating clamp 12 is employed to move the dosage calibration container 24 and the vial 2 back to the original position of dosage calibration container 24. The robotic manipulating clamp 12 then holds a neck of the vial 2 and retrieves the vial 2 from the dosage calibration container 24 and moves the vial 2 to the vial container 21 in order to perform subsequent radiopharmaceuticals withdrawal and dispensing processes.

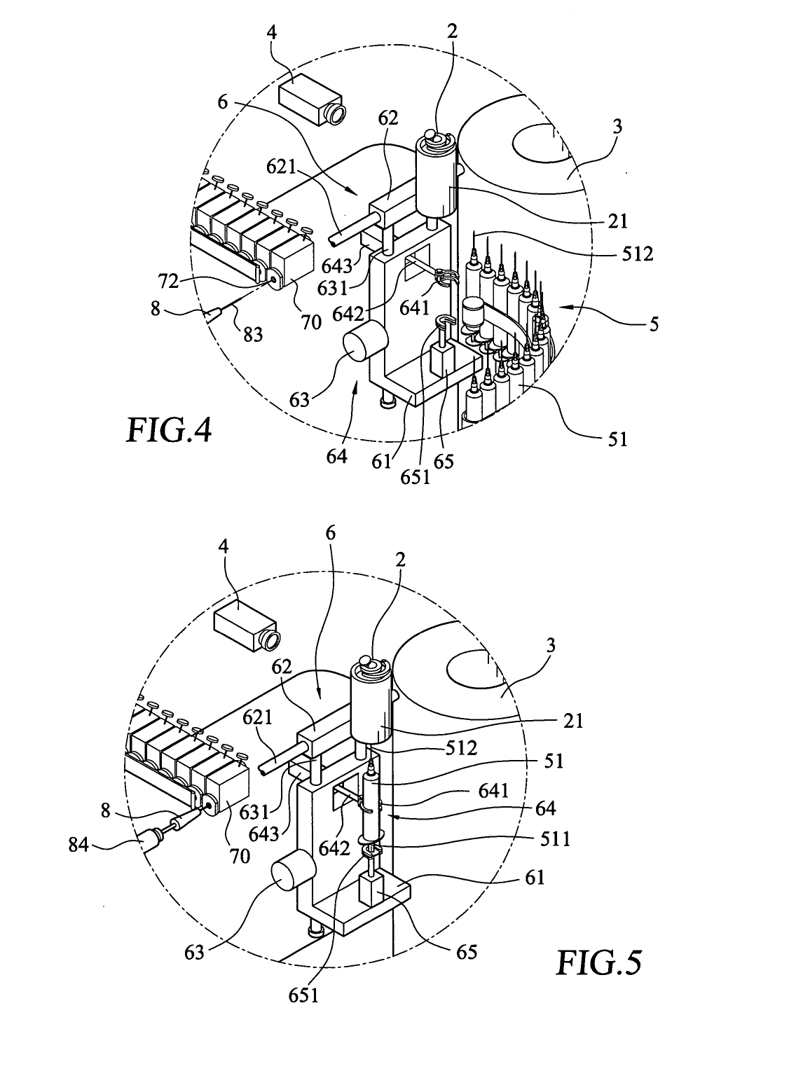

Referring to FIG. 4, inside the radiation-shielded hermetic chamber 1, disposed at a position adjacent a bottom of the vial container 21 is a disposable syringe module 5, which comprises a plurality of disposable syringes 51 arranged in a line and a saline water cartridge container 7 comprises a plurality of saline water cartridges 70 arranged in a line.

A movable dispensing and injection mechanism 6 is arranged inside the radiation-shielded hermetic chamber 1 at a position adjacent to the disposable syringe module 5 and the vial container 21 for controlling radiopharmaceuticals dispensing operation of a selected one of the disposable syringes 51 and for controlling movement of the selected disposable syringe between a dispensing position and an injection position.

Referring to FIGS. 4 and 5 simultaneously, the movable dispensing and injection mechanism 6 comprises a support carrier 61, a horizontal transportation mechanism 62, a vertical transportation mechanism 63, a clamping and releasing mechanism 64, and a plunger driving mechanism 65. The horizontal transportation mechanism 62 moves the support carrier 61 in a horizontal direction along at least one horizontal rail 621 and the vertical transportation mechanism 63 moves the support carrier 61 in a vertical direction along at least one vertical rail 631.

The clamping and releasing mechanism 64 comprises a pick-up arm 641, an extension bar 642, and a clamping and releasing controller 643 for controlling clamping and releasing a selected one of the disposable syringes 51. When the extension bar 642 is driven by the clamping and releasing controller 643 to extend outward, the pick-up arm 641 is opens to pick up one disposable syringe 51 (as shown in FIG. 5).

The plunger driving mechanism 65 can be a motor and a pneumatic cylinder, which is mounted on the support carrier 61 for driving an upward forwarding operation and a downward radiopharmaceuticals withdrawal operation of the plunger 511 of the selected disposable syringe 51. The plunger driving mechanism 65 may further comprise a pressure sensor 651 for detecting the pressure applied to the plunger 51 of the disposable syringe 51.

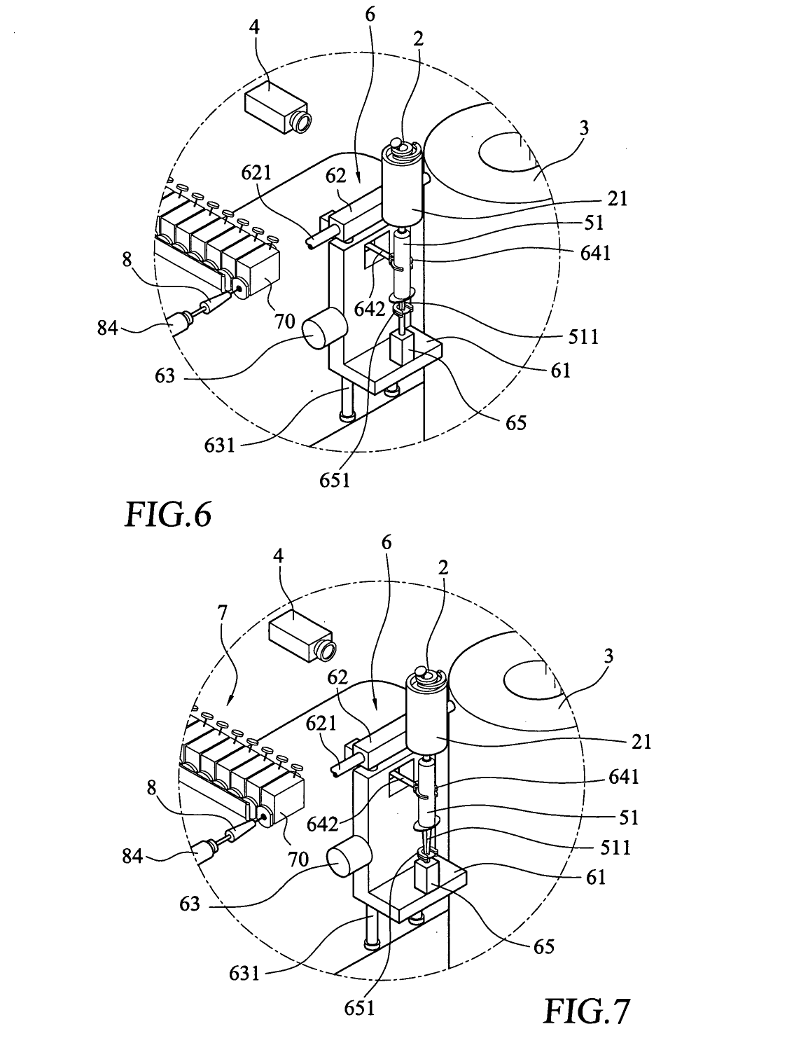

Once the selected disposable syringe 51 is held by the pick-up arm 641, which moves back to the original retreated position, the disposable syringe 51 is fixed in the radiopharmaceuticals dispensing position. At this time, the vertical transportation mechanism 63 drives the whole support carrier 61 to displace upwards along the vertical rail 631, thereby causing a needle 512 atop the disposable syringe 51 to penetrate into a bottom of the vial 2 (as shown in FIG. 6). Thereafter, under the driving and control of the plunger driving mechanism 65, the plunger 51 of the disposable syringe 51 is pulled downward (as illustrated in FIG. 7), whereby a predetermined amount of radiopharmaceuticals is withdrawn from the vial 2.

After the withdrawal operation of radiopharmaceuticals described above, the vertical transportation mechanism 63 causes the support carrier 61 to move downward along the vertical rail 631, which causes the disposable syringe 51 to displace downward and disengaging the needle 512 from the bottom of the vial 2. Thereafter, under the condition that the disposable syringe 51 is moved by being driven by the horizontal transportation mechanism 62, the disposable syringe 51 displaces to the injection position (that is a position adjacent to the injection needle insertion hole 11 of the radiation-shielded hermetic chamber 1).

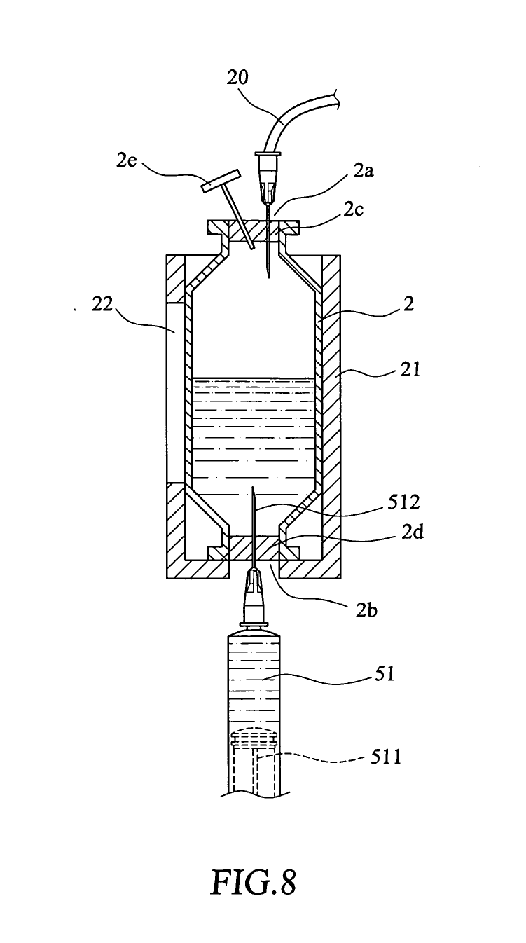

In a preferred embodiment of the present invention, the vial 2 has a two-open-end structure (referring to FIG. 8), wherein the main body of the vial 2 has a top opening 2a and a bottom opening 2b, in which top plug body 2c and a bottom plug body 2d are fit respectively. Radiopharmaceuticals is supplied into the vial 2 by the radiopharmaceuticals supply tube 20 that extends through the top plug body 2c, while the needle 512 of the disposable syringe 51 can penetrate through the bottom plug body 2d for withdrawal of the radiopharmaceuticals contained in the via 2. The top plug body 2c of the top opening 2a of the vial 2 further comprises a filter 2e inserted therethrough whereby negative pressures may not induced inside the vial 2 when the radiopharmaceuticals inside the vial 2 is withdrawn.

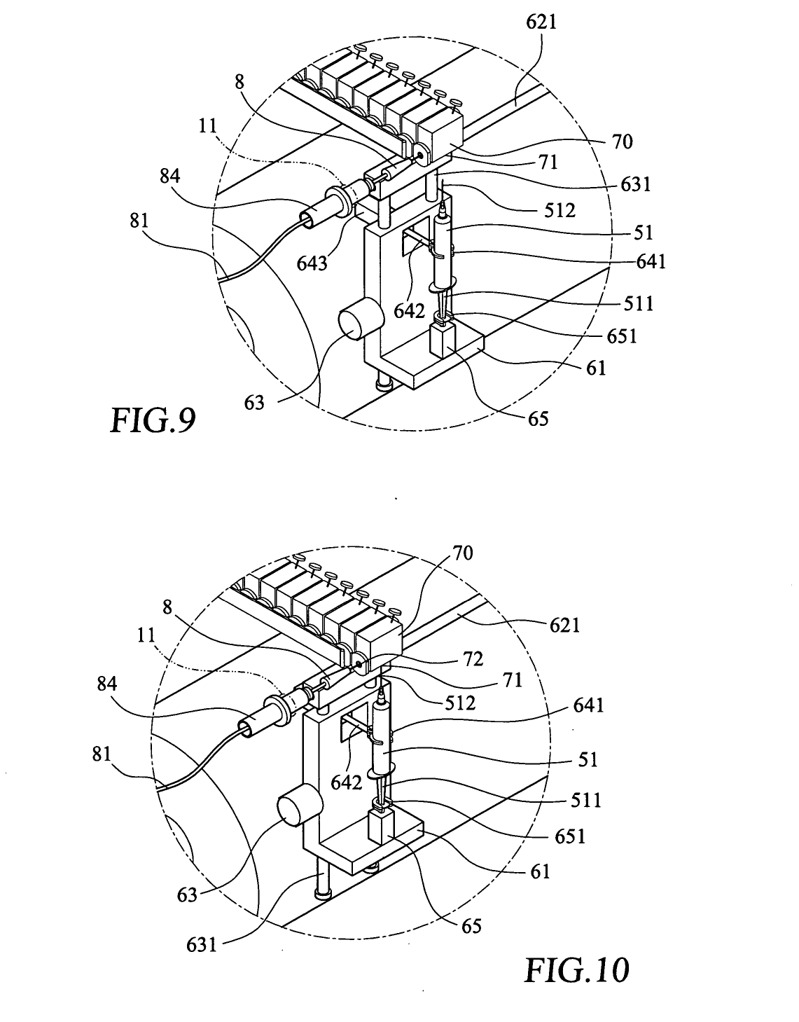

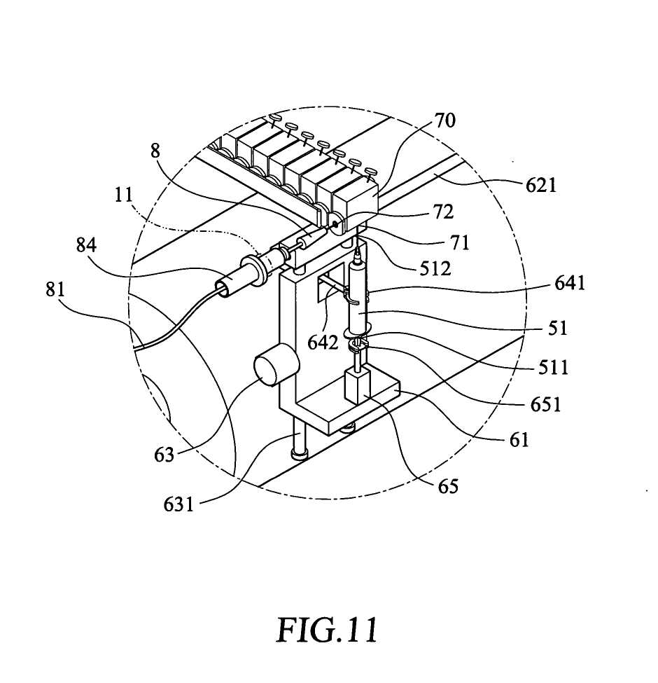

FIG. 9 shows an enlarged partial perspective view illustrating the condition when the disposable syringe 51 is driven by the horizontal transportation mechanism 62 of the movable dispensing and injection mechanism 6 to move to the injection position, but the needle 512 does not penetrate through a radiopharmaceuticals injection end 71 of the saline water cartridge 70. FIG. 10 shows an enlarged partial perspective view illustrating the condition when the disposable syringe 51 is located in the injection position and the needle 512 penetrates through the radiopharmaceuticals injection end 71 of the saline water cartridge 70. FIG. 11 shows an enlarged partial perspective view illustrating the condition when the disposable syringe 51 is located in the injection position, the needle 512 penetrates through the radiopharmaceuticals injection end 71 of the saline water cartridge 70, and the plunger 511 of the disposable syringe 51 is pushed upward.

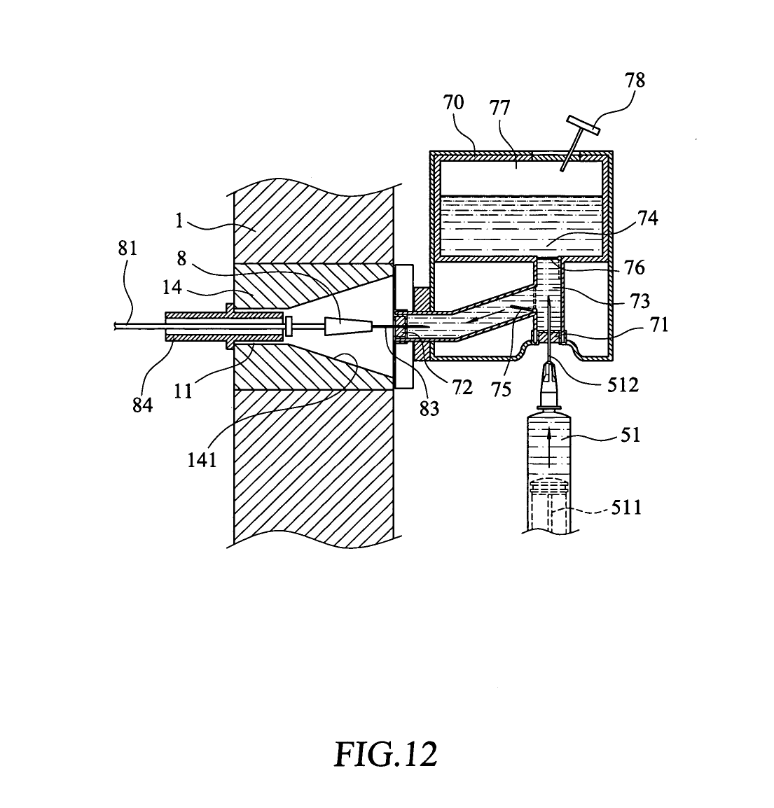

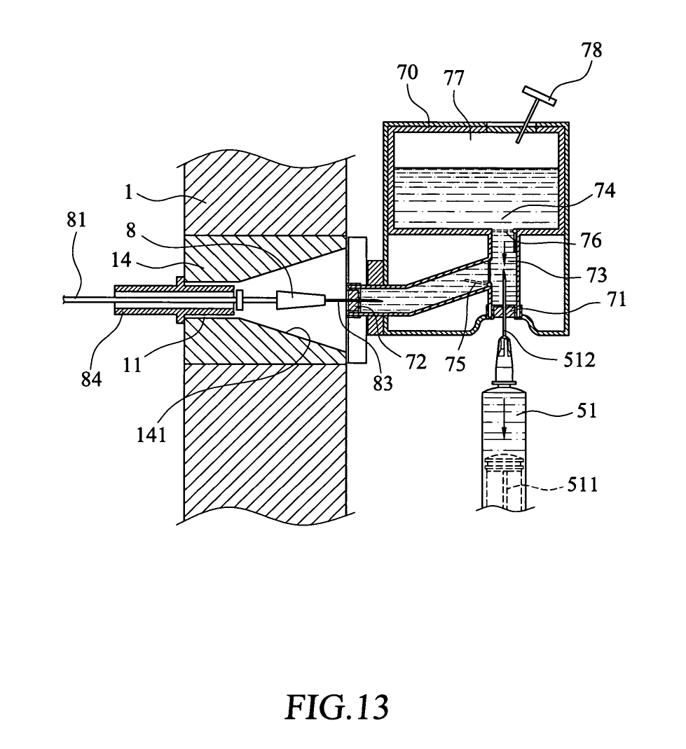

FIG. 12 shows a cross-sectional view illustrating that condition when the needle 512 of the disposable syringe 51 penetrates through the radiopharmaceuticals injection end 71 of the saline water cartridge 70 and the plunger 511 of the disposable syringe 51 is pushed upward. FIG. 13 shows a cross-sectional view illustrating the condition when the needle 512 of the disposable syringe 51 penetrates through the radiopharmaceuticals injection end 71 of the saline water cartridge 70 and the plunger 511 of the disposable syringe 51 is pulled downward. The saline water cartridge module 7 is arranged inside the radiation-shielded hermetic chamber 1 at the injection position that is adjacent to the injection needle insertion hole 11.

As shown in FIGS. 12 and 13, a radiopharmaceuticals discharge end 72 of the saline water cartridge 70 allows for insertion of a needle 83 of a insertion section 8. The insertion section 8 has a rear end to which a tube 81 is connected. The tube 81 extends through the injection needle insertion hole 11 of the radiation-shielded hermetic chamber 1 and is connected to a needle 82 (also see FIG. 1) that is inserted into a patient's body.

At a suitable position of the tube 81, a conventional and rotation-operated three-way valve 811 (see FIG. 1) is provided, having an end connected to the insertion section 8 by the tube 81 and another end connected through a terminal filter 812 to the patient needle 82. A saline water syringe 813 is inserted into a top face of the three-way valve 811 to fill the tube 81 with saline water before injection is performed, which avoids air existing in the tube 81 when injection is performed.

The structure of the saline water cartridge 70 comprises a radiopharmaceuticals injection end 71, a radiopharmaceuticals discharge end 72, an internal passage 73, and a saline water reservoir outlet end 74. A first one-way membrane valve 75 is arranged in the internal passage 73 at the radiopharmaceuticals discharge end 72 and a second one-way membrane valve 76 is arranged at the saline water reservoir outlet end 74.

The saline water cartridge 70 forms a saline water reservoir 77 therein, in which saline water is stored. The saline water reservoir 77 is connected to the internal passage 73 by the saline water reservoir outlet end 74 to allow the saline water to flow into the internal passage 73 of the saline water cartridge 70. In addition, in practical operation, an air filter 78 is inserted into the top face of the saline water reservoir 77 to prevent the induction of negative pressure inside the saline water reservoir 77 when the saline water inside the saline water reservoir 77 is withdrawn.

When the needle 83 of the insertion section 8 is inserted into the radiopharmaceuticals discharge end 72 of the saline water cartridge 70, the insertion section 8 passes through the injection needle insertion hole 11 of the radiation-shielded hermetic chamber 1 to allow the needle 83 to penetrate through the radiopharmaceuticals discharge end 72 of the saline water cartridge 70.

To carry out penetration of the insertion section 8 into the radiopharmaceuticals discharge end 72 of the saline water cartridge 70, an auxiliary tube 84 is employed, which allows the needle 83 of the insertion section 8 to easily penetrate into the radiopharmaceuticals discharge end 72. To prevent radiation from emitting through the injection needle insertion hole 11 of the radiation-shielded hermetic chamber 1, an insert 14 made of tungsten material is inserted into the injection needle insertion hole 11. The insert 14 has an internal wall forming an inclined surface 141.

After the movable dispensing and injection mechanism 6 moves the disposable syringe 51 to the injection position (that is a position adjacent to the injection needle insertion hole 11 of the radiation-shielded hermetic chamber 1), the vertical transportation mechanism 63 drives the whole support carrier 61 to displace upward along the vertical rail 631, with which the needle 512 of the disposable syringe 51 is caused to penetrate through the radiopharmaceuticals injection end 71 of the saline water cartridge 70. Thereafter, the plunger 511 of the disposable syringe 51 is driven and controlled by the plunger driving mechanism 65 to displace and push upward whereby the radiopharmaceuticals that is previously withdrawn into and currently contained in the disposable syringe 51 is injected into the radiopharmaceuticals injection end 71 of the saline water cartridge 70.

At this moment, the first one-way membrane valve 75 of the saline water cartridge 70 is in an open condition (as shown in FIG. 12), while the second one-way membrane valve 76 is in a closed condition. Thus, radiopharmaceuticals can be fed from the internal passage 73 to the radiopharmaceuticals discharge end 72 and supplied through the needle 83, the insertion section 8, the tube 81, the three-way valve 811, and the terminal filter 812 to the needle 82 inserted into the patient's body.

When the injection operation of the radiopharmaceuticals is completed, the plunger 511 of the disposable syringe 51 is driven and controlled by the plunger driving mechanism to move downward for carrying out at least one flushing cycle. At this moment, the first one-way membrane valve 75 of the saline water cartridge 70 is in a closed condition, while the second one-way membrane valve 76 is in an open condition (as shown in FIG. 13). Thus, saline water in the saline water reservoir outlet end 74 can be withdrawn by the disposable syringe 51 through the saline water reservoir outlet end 74 and the radiopharmaceuticals injection end 71.

After the withdrawal operation of the saline water, the disposable syringe 51 is driven by the plunger driving mechanism 65 to push upward again and the saline water is injected into the radiopharmaceuticals injection end 71 of the saline water cartridge 70. At this moment, the first one-way membrane valve 75 is in an open condition, while the second one-way membrane valve 76 is in a closed condition. Thus, the saline water is allowed to flow through the internal passage 73 to the radiopharmaceuticals discharge end 72 and supplied through the needle 83, the insertion section 8, the tube 81, the three-way valve 811, and the terminal filter 812 to the needle 82 inserted into the patient's to thereby effect flushing of radiopharmaceuticals.

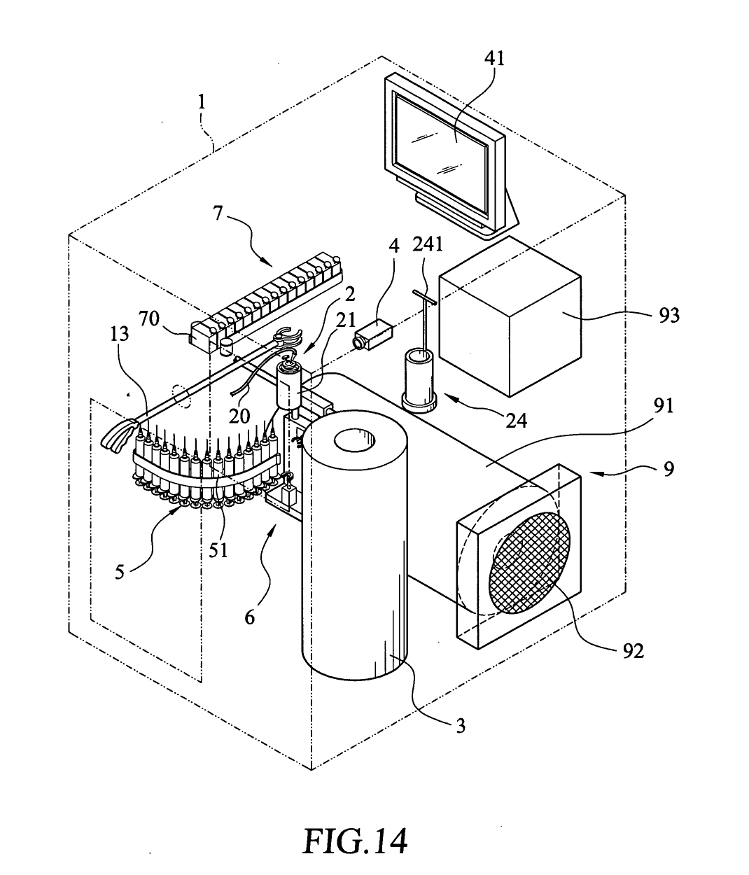

Referring to FIG. 14, which shows a rear-side perspective view of the radiation-shielded radiopharmaceuticals dispensing and injection system in accordance with the present invention, the radiation-shielded hermetic chamber 1 further comprises an air filtration device 9, comprising at least one filter 91 and a fan 92 for maintaining cleanness of air and providing positive pressure inside the radiation-shielded hermetic chamber 1. The radiation-shielded hermetic chamber 1 may also comprise a sampling device 93, which comprises for example a sampling syringe and a sampling pump for sampling the radiopharmaceuticals contained in the vial 2 for quality control purposes.

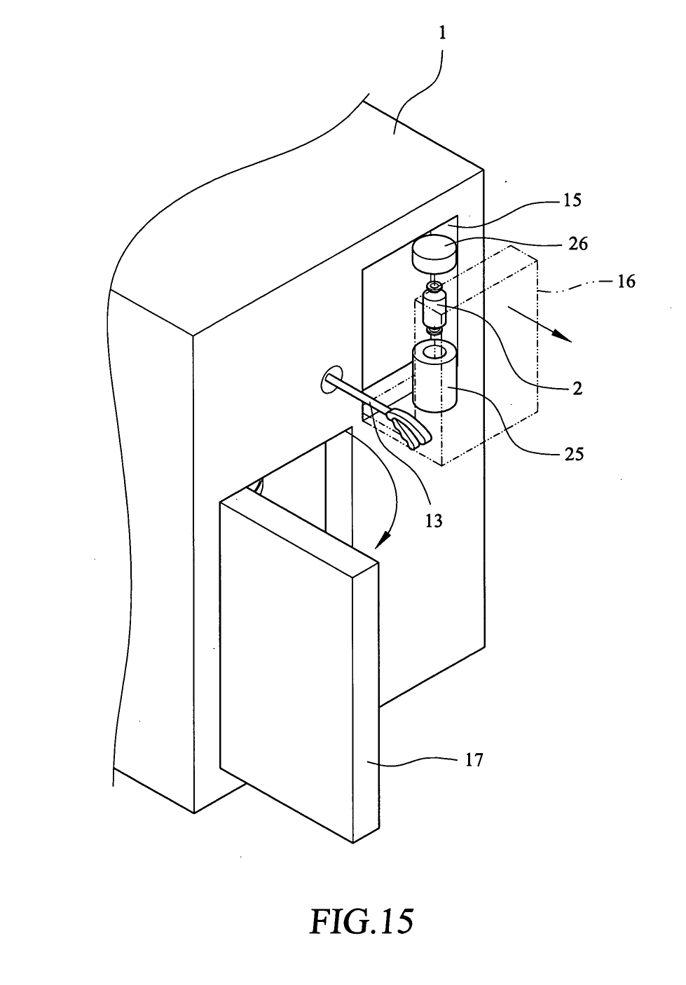

Referring to FIG. 15, which shows a perspective view of the radiation-shielded radiopharmaceuticals dispensing and injection system in accordance with the present invention in which a withdrawable carrier, a vial access opening zone, and a door are arranged, as described above, the radiopharmaceuticals inside the vial 2 is supplied into the vial 2 from a laboratory or dosing room through the radiopharmaceuticals supply tube 20. This can certainly be replaced by conveyance with a robotic manipulator or manually.

When the vial 2 is moved out of the radiation-shielded hermetic chamber 1 for conveyance of the radiopharmaceuticals or when the radiopharmaceuticals is manually moved into the radiation-shielded hermetic chamber 1, the radiopharmaceuticals must be contained in a radiation-shielded and sealed container. Thus, the radiation-shielded hermetic chamber 1 of the present invention is further provided with a vial access opening zone 15, which allows for receipt of the withdrawable carrier 16 into the hermetic chamber and allows the withdrawable carrier 16 to be withdrawn through the vial access opening zone 15. The withdrawable carrier 16 is made of radiation-shielding material. When the withdrawable carrier 16 is moved into the radiation-shielded hermetic chamber 1, the withdrawable carrier 16 completely blocks the vial access opening zone 15.

For example, to manually convey the radiopharmaceuticals into the radiation-shielded hermetic chamber 1, the vial 2 in which the radiopharmaceuticals 2 is stored is positioned into a radiation-shielded conveyance container 25 and sealed with a lid 26. The whole container 21 is placed in the withdrawable carrier 16 that is withdrawn outward and the withdrawable carrier 16 is then pushed into the radiation-shielded hermetic chamber 1 to allow an operator to pick up or move the vial 2 with the manipulating clamp 12.

The radiation-shielded hermetic chamber 1 is also provided with a door 17, which is also made of radiation-shielding material, whereby access through the door is provided for maintenance.

Although the present invention has been described with reference to the preferred embodiment thereof, it is apparent to those skilled in the art that a variety of modifications and changes may be made without departing from the scope of the present invention which is intended to be defined by the appended claims.

1. A radiation-shielded dispensing and injection system for radiopharmaceuticals, comprising:
a radiation-shielded hermetic chamber, forming a closed, radiation-shielded interior space, the radiation-shielded hermetic chamber also forming at least one injection needle insertion hole;
a vial containing radiopharmaceuticals therein;
at least one saline water cartridge arranged inside the hermetic chamber at an injection position adjacent to the injection needle insertion hole, the saline water cartridge forming an internal passage, a radiopharmaceuticals discharge, and a radiopharmaceuticals injection end;
at least one disposable syringe; and
a movable dispensing and injection mechanism for controlling radiopharmaceuticals dispensing operation of the syringe, and controlling movement of the syringe between a radiopharmaceuticals dispensing position and the injection position;
wherein when the movable dispensing and injection mechanism moves the syringe to the radiopharmaceuticals dispensing position, the syringe withdraws a predetermined amount of radiopharmaceuticals from the vial and when moved to the injection position, the syringe injects the withdrawn radiopharmaceuticals into the radiopharmaceuticals injection end of the saline water cartridge.

2. The radiation-shielded dispensing and injection system for radiopharmaceuticals as claimed in claim 1 further comprising a dosage calibrator arranged inside the radiation-shielded hermetic chamber for detecting radioactivity of the radiopharmaceuticals contained in the vial.

3. The radiation-shielded dispensing and injection system for radiopharmaceuticals as claimed in claim 1, wherein the movable dispensing and injection mechanism comprises:
a support carrier;
a horizontal transportation mechanism which drives the support carrier in a horizontal direction along a horizontal guiding mechanism;
a vertical transportation mechanism which drives the support carrier in a vertical direction along a vertical guiding mechanism;
a clamping and releasing mechanism for clamping and releasing the disposable syringe; and
a plunger driving mechanism mounted on the support carrier for driving a plunger of the disposable syringe to displace upward or to withdraw the radiopharmaceuticals.

4. The radiation-shielded dispensing and injection system for radiopharmaceuticals as claimed in claim 3 further comprising a pressure sensor arranged below the plunger driving mechanism for detecting pressure applied to the plunger of the disposable syringe.

5. The radiation-shielded dispensing and injection system for radiopharmaceuticals as claimed in claim 1 further a radiopharmaceuticals level monitoring device arranged inside the radiation-shielded hermetic chamber for monitoring liquid level of the radiopharmaceuticals filled in the vial.

6. The radiation-shielded dispensing and injection system for radiopharmaceuticals as claimed in claim 1 further comprising an air filtration device comprising a filter and a fan for maintaining cleanness of air and providing a positive pressure inside the radiation-shielded hermetic chamber.

7. The radiation-shielded dispensing and injection system for radiopharmaceuticals as claimed in claim 1, wherein the radiation-shielded hermetic chamber further comprises a vial access opening zone through which a withdrawable carrier is received in the hermetic chamber, and which allows the withdrawable carrier to be withdrawn outward through the vial access opening zone.

8. The radiation-shielded dispensing and injection system for radiopharmaceuticals as claimed in claim 1 further comprising a radiation-shielded container for accommodating the vial whereby when the vial is moved out of the radiation-shielded hermetic chamber for conveyance of the radiopharmaceuticals, the vial is shielded by the radiation-shielded container.

9. The radiation-shielded dispensing and injection system for radiopharmaceuticals as claimed in claim 1 further comprising a manipulating clamp extending into the radiation-shielded hermetic chamber to serve as a tool for manual operation.

10. The radiation-shielded dispensing and injection system for radiopharmaceuticals as claimed in claim 1, wherein the saline water cartridge comprises a saline water reservoir which is connected to the internal passage of the saline water cartridge through a saline water reservoir outlet end to allow saline water to flow into the internal passage.
